(19) United States
(12) Patent Application Publication
(10) Pub. No.: US 2025/0177778 A1
(43) Pub. Date: Jun. 5, 2025

(54) SYNTHETIC PLANNING OF ROBOTIC ARC THERAPY (SPRAT)

(71) Applicant: Michael Weil, Half Moon Bay, CA (US)

(72) Inventor: Michael Weil, Half Moon Bay, CA (US)

(21) Appl. No.: 18/969,271

(22) Filed: Dec. 5, 2024

Related U.S. Application Data
(60) Provisional application No. 63/606,354, filed on Dec. 5, 2023.

(51) Int. Cl. A61N 5/10

(52) U.S. Cl. CPC A61N 5/1031; A61N 5/1083

(57) ABSTRACT

The present invention relates to x-ray treatment of body lesions. The system acquires images and plans therapy with a non-invasive, kV radiation source. It employs a treatment plan obtained via an artificial intelligence (AI) enhanced Cone Beam Computed Tomography (synthetic CT, sCBCT), wherein reconstructed images are the basis of an optimized plan. Focused kV beams are used to definitively treat, or to enhance immune modulators, to destroy tumors. Distributed external beam entry points yield a significant advantageous dose at depth, wherein intersecting beams accumulate in a restricted focus. The output is planned from a synthetic CBCT obtained in real time at the start of a procedure. Computer optimization selects x-rays traveling towards a pathological location and quenches those striking undesired locations. Beam localization is done by moving a robot-mounted x-ray source around a patient to distribute dose to clinically tolerable levels. A second robot manipulates a flat-panel detector for image capture opposite the source. This invention optimizes delivery of kV beams to deep lesions via sCBCT guidance.

### RELATED U.S. APPLICATION DATA

This application claims priority of provisional application 63/606,354 with a filing date of Dec. 5, 2023.

### REFERENCES CITED

### U.S. Patent Documents

U.S. Pat. No. 7,481,758 January 2009 Weil and Morris
Ser. No. 18/099,668 January 2023 Weil

### Other Publications

Bazalova-Carter, et al., “Feasibility of external beam radiation therapy to deep-seated targets with kilovoltage x-rays,” Med Phys 44:597, 2017, AIP.

### FIELD OF THE INVENTION

The claimed invention relates generally to cancer therapy and imaging of lesions that are life-threatening. The system's components optimize distribution of externally applied, therapeutic kilovoltage (kV) x-rays to deliver situation-specific curative dosimetry. The treatment and imaging are performed with a single kV source whose output is localized to deposit lethal energy within a pathologic lesion. Efficacious therapy is derived by aptly adjusting delivery parameters to minimize relevant cost functions in real time. The invention utilizes synthetic imaging and precisely overlaid beam plans for the targeted anatomy that are delivered via robotic controls of an x-ray source and detector. Multiple circumscribing robots safely implement imaging and optimal therapeutic mechanisms.

### BACKGROUND OF THE INVENTION

Radiotherapy, also known as radiation therapy, is a medical treatment that uses high-energy radiation to target and destroy cancer cells in the body. It is one of the primary methods used in the treatment of cancer, either as a standalone treatment or in combination with surgery, immunotherapy or chemotherapy. The process of radiotherapy entails the following elements:

1. Treatment Planning: Before starting radiotherapy, a team of medical professionals, including radiation oncologists and medical physicists, carefully plan the treatment. This involves a series of steps to ensure that the radiation is delivered precisely to the tumor while minimizing damage to surrounding healthy tissues. Using specialized dosimetry software, the medical team develops a treatment plan that determines the appropriate radiation dose, beam angles, and techniques to deliver the radiation. The goal is to maximize the dose to the tumor while minimizing exposure to healthy tissues.
(i) Prior to Treatment Simulation: During simulation, imaging techniques such as computed tomography (CT) scans, and when appropriate magnetic resonance imaging (MRI) and positron emission tomography (PET), are used to determine the exact location, size, and shape of the tumor. Patients may be asked to lie in a specific position or wear immobilization devices to ensure consistent positioning during treatment.
(ii) Definition of Target Volume: The radiation oncologist, in collaboration with the treatment planning team, outlines the target volume, which includes the tumor and any nearby lymph nodes at risk of containing cancer cells. They also delineate critical structures, such as organs or tissues that need to be protected during treatment.

2. Radiation Delivery: Once the treatment plan is finalized, the actual radiotherapy sessions can begin. The common methods used for delivering radiation are:
(i) External Beam Radiation Therapy: This is the most common form of radiotherapy. Conventionally it involves using a machine called a linear accelerator that generates high-energy, megavoltage (MV) x-rays or electrons. The patient lies on a treatment table, and the machine rotates around them, delivering the radiation from different angles. The treatment is painless, and each session typically lasts a few minutes. The course of therapy can be a single targeted session for small tumors or may last several months for large volumes.
(ii) Intensity-Modulated Radiation Therapy (IMRT): IMRT is an advanced technique that allows for precise control over the radiation intensity delivered to different parts of the tumor. It uses computer-controlled multileaf collimators to vary the intensity of the radiation during the rotation of the beams, sculpting the dose distribution to match the shape of the tumor. This helps spare nearby healthy tissues.
(iii) Image-Guided Radiation Therapy (IGRT): IGRT involves using imaging techniques, such as CT scans or x-rays or MRI, before or during each treatment session to ensure accurate tumor targeting. This helps compensate for any changes, during the course of treatment, in the tumor's position or shape, as well as the patient's internal anatomy.

3. Treatment Sessions and Monitoring: Radiotherapy is typically delivered in multiple sessions, called fractions, spread over several weeks. The total number of sessions depends on the type, location, and stage of cancer. Each session is carefully monitored by a radiation therapist, who operates the equipment from a control room. They can see and hear the patient throughout the session via leaded windows and closed-circuit television (CCTV) and can communicate with them via an intercom.

4. Side Effects and Supportive Care: Radiotherapy can cause side effects, which vary depending on the treated area and the individual. Common side effects include fatigue, skin reactions (e.g., redness, itching), hair loss in the treatment area, and temporary changes in the body's functions. The medical team provides supportive care and may recommend medications, creams, or lifestyle adjustments to manage these side effects.

5. Follow-up Care: After completing the radiotherapy sessions, patients typically undergo regular follow-up appointments with their radiation oncologist. These appointments involve monitoring the treatment's effectiveness, assessing any lingering side effects, and addressing any concerns the patient may have.

It is important to note that the process of radiotherapy may vary depending on the specific cancer type, stage, and individual patient characteristics. The treatment is always tailored.

### SUMMARY OF THE INVENTION

Radiotherapy is a highly intricate and specialized medical treatment that utilizes ionizing radiation to target and destroy cancer cells. Its infrastructure involves a comprehensive network of components and processes, working in unison to ensure safe and effective treatment delivery. The complex infrastructure of radiotherapy includes:

1. Treatment Planning System (TPS): Sophisticated software integrates patient-specific data, including medical images (CT, MRI, PET scans), to create a customized treatment plan of specific beam geometry and x-ray characteristics. A TPS calculates the optimal radiation dose, beam angles, and other parameters, considering the tumor's location and surrounding healthy tissues.

2. Linear Accelerator (Linac): The Linac is the primary device responsible for generating and delivering the therapeutic megavoltage (MV) radiation. Its highly inticate design produces high-energy x-rays or electron beams that are precisely directed towards a tumor site. Linacs are equipped with sophisticated control systems, ensuring accurate beam shaping, intensity modulation, and positioning. Modern Linacs also incorporate image-guided radiation therapy (IGRT) capabilities, enabling real-time imaging during treatment.

3. Treatment Delivery Systems: The treatment delivery system encompasses various components that assist in delivering the radiation accurately. This includes multi-leaf collimators (MLCs), which shape the radiation beam to conform to the tumor's shape, and compensators, which help modulate the dose distribution. Advanced techniques like intensity-modulated radiation therapy (IMRT) and volumetric modulated arc therapy (VMAT) allow for precise dose sculpting, increasing treatment efficacy while minimizing damage to healthy tissues.

4. Image-Guidance Systems: Image-guidance systems play a crucial role in ensuring the accuracy of radiation delivery. They utilize imaging modalities, such as cone-beam computed tomography (CBCT) or kilovoltage imaging, to acquire real-time images of the patient's anatomy before or during treatment. These images are compared to the reference images from the treatment plan, allowing clinicians to make necessary adjustments and align the patient position accurately.

5. Quality Assurance (QA) and Dosimetry: QA processes are essential to maintain the safety and precision of radiotherapy treatments. Dosimetry refers to the measurement and verification of radiation doses delivered to the patient. Specialized equipment, such as ionization chambers and diode detectors, is used to ensure the accuracy of the radiation dose. Regular calibration, quality control checks, and adherence to strict protocols are integral to the QA process.

6. Treatment Verification and Recordkeeping: Radiotherapy infrastructure incorporates comprehensive systems for treatment verification and recordkeeping. Patient-specific treatment data, including images, treatment plans, and delivery parameters, are stored and managed in electronic medical records (EMR) or radiation oncology information systems (ROIS). These systems facilitate seamless communication and coordination among the treatment team, ensuring accurate and up-to-date information for each patient.

7. Radiation Protection and Safety: Radiotherapy facilities must adhere to stringent safety measures to protect both patients and healthcare professionals from unnecessary exposure to radiation. Special shielding, such as 2-meter-thick lead-lined walls and doors, are used to contain the radiation within the treatment room. Strict protocols, training, and safety checks ensure that radiation doses are accurately delivered and at the same time minimizing the risk to surrounding personnel.

The infrastructure of radiotherapy is continuously evolving with advancements in technology and treatment techniques. This ongoing progress aims to improve treatment outcomes, enhance patient experience, and further enhance the precision and effectiveness of radiotherapy in combination with other prescription modalities to better treat cancer.

### BRIEF DESCRIPTION OF THE FIGURES

The construction and usage of embodiments will become readily apparent from consideration of the following specification as illustrated in the accompanying drawings, in which like reference numerals designate like parts, and wherein:

FIG. 1 is a diagram illustrating a system according to some embodiments, which treats using coplanar beamlets in a focused distribution to deliver an energy dose localized to a lateral position in the body;

FIG. 2 illustrates elements of arrayed source positions, which improve dosimetry to the body, wherein the pattern of source output is effectively directed towards a pathologic location and energy traveling towards critical structures are quenched; and

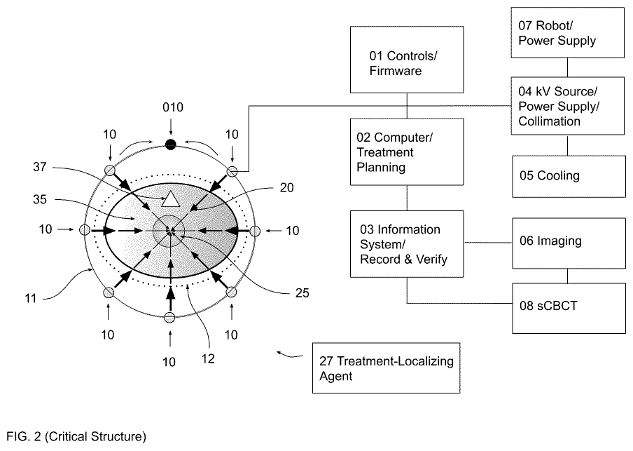

FIG. 3 is a simplified perspective view of arrayed source positions in and out of plane as well as delivery components according to some embodiments, which improve dosimetry to the body.

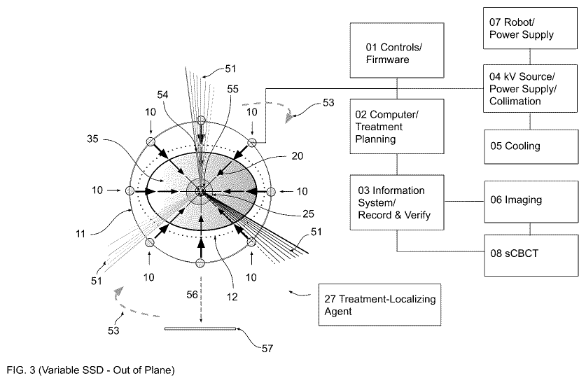

### DETAILED DESCRIPTION OF THE INVENTION

The construction and usage of embodiments will become readily apparent from consideration of the following specification as illustrated in the accompanying drawings, in which like reference numerals designate like parts, and wherein:

In clinical medical practice, therapeutic x-rays are evaluated and prescribed based on dose distributions generated in computer models of tomographic kilovoltage (kV) images.

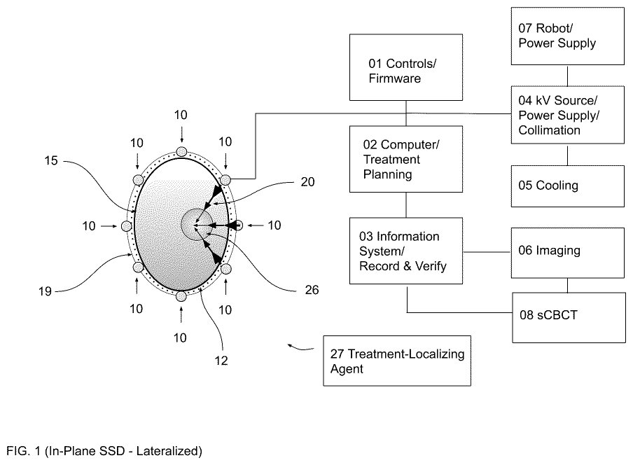

The present invention recites the usefulness of implementing and tracking key treatment workflow tasks (FIG. 1), e.g., i) Imaging (CBCT) 06, ii) Segmentation, iii) Planning+Optimization 02, iv) Registration, and v) Treatment (“I-SPORT”). Imaging 06 and Planning 02 bracket the target delineation step. This Segmentation can be addressed manually or automated to enable the robotic delivery of the Treatment wherein an array of positions of the ionizing energy source are distinguished by optimized geometries and times. Beam collimation 04, following delivery paths originating in a treated tumor, can be performed with a pinhole, an iris, multiple geometries or multi-leaf apertures. The plans and optimized treatment 02 simulations can be validated and verified in phantoms using radiochromic film dosimetry. Incorporation of other verification technology 03, such as scintillation detection as manufactured by Medscint (Hyperscint dosimeter), can be employed and thereby provide real-time, non-invasive x-ray source beam output measurement at the beam exit from the source.

1. Room and source shielding; (i) The system requires a shielded operator area with adjustment for 225 kV, (ii) such a Comet source operating at full power, has leakage radiation of 10 mSv/h @ 1 meter <Retrieved from the Internet>. Thus it will require ˜5 kg of lead to reduce scatter to 0.88 mGy/h @ 1 meter [(vice 100 mR exposure) “Leakage radiation from . . . ”]<Retrieved from the Internet>, (iii) The shielded room must meet the spatial requirements for 2-robot operation, and construction factors.

2. Collimator 04 and aperture mounting; To align a collimated beam 04 and laser/light beams (yielded by collimator), it is necessary to have a properly machined mounting fixture for a commercial collimator 04, e.g., Huestis-type <Retrieved from the Internet>. Such tolerances are taken into account in the collimator harness design and include but are not limited to the methods of testing and verification of light/laser/x-ray beam correspondence.

3. Implementation of treatment plan 02 by system controls 01; The system includes a usable GUI (graphic user interface) to set up from an isocenter and maintain targeted delivery, which along with firmware to control robots, also enables non-coplanar source movement. The treatment plan 02 employs an artificial intelligence (AI) enhanced CBCT (synthetic CT, sCBCT 08), wherein a reconstructed image is the basis of an optimized treatment plan 02. This model then informs the robot controls 01 to align the x-ray source according to that plan 02. The plan and required robotic movements 07 are based on a 3D coordinate system bound to a shielded room. Such spatial delineation is used to orient a body or targeted lesion with respect to the fixed coordinate system. In the present invention, the treatment planning software 02 calculates a dose distribution around the X-, Y-, Z-coordinates of the focal spot in stereotactic space (including azimuth and elevation angles). In the instance of treating with a pencil beam, such information goes to the robot 07 holding the x-ray source. Additionally, when treating with a multi-leaf collimator, all three Euler angles are taken into account. Implementation of clinically beneficial x-ray therapy thus requires designing the corresponding robot firmware 01 to actualize the parameters generated by the TPS 02. These numbers are thus passed from one software unit to another.

The x-ray source control 01 includes collimation 04 that may be capable of supporting a pencil beam or more typically an iris or multileaf occlusion wherein treatment requires a beam aperture of a customized shape. The planned dosimetry for the latter entails multiple source positioning and specific exposition techniques for every source position. Thus the procession of the source around the targeted lesion encompasses implementing step-by-step treatment parameters wherein knowledge of the final dose distribution is mandatory.

The treatment planning software 02 calculates and defines XYZ coordinates of the focal spot in 3D space (where the coordinate system is most likely bound to a room) and, exempli gratia, azimuth and elevation angles, in the case of the pencil treating beam, or all three Euler angles in the case of multi-leaf collimation. Such information goes to the x-ray source 04-holding robot 07 and the corresponding robot firmware 01. Typically, a treatment requires a beam aperture of a certain shape. This entails multiple source positioning and certain exposition techniques for every position.

Preferably pre-treatment with a modality to induce necrosis such as by the present invention with concentric, converging x-ray beams at ablating doses can be permissive for induction of antitumor immunity. Thus, this enables systemic infusion of an immunomodulator, or other compound, but limits its interactions to a localized volume of interest. Targeting a lesion within a body by employing volumetrically discrete energy dose deposition to beneficially manipulate the lesion with a therapeutic compound therein, as recited in the present invention, comprises a treatment-localizing and enhancing agent 27. This is important in the case of inducing beneficial therapeutic, antitumor immunity with GM-CSF, where localized cytokine delivery to necrotic debris is thought to be efficacious and has been described (U.S. Pat. No. 7,481,758), while systemic distribution is undesirable since it can induce immune suppression and other side effects (Weil and Morris, unpublished). The Synthetic Planning of Robotic Arc Therapy (SPRAT), as disclosed herein, is an effective modality for inducing such adjuvant and definitive necrosis.

What is claimed is:

1. A system comprising:
a. a mechanically distributed energy radiating source, which is external and around a body, or head, to treat and image pathologic lesions;
b. wherein the radiated energy is ionizing, ablating electromagnetic kV ranging from 100-350 kVp;
c. wherein the radiated energy source's orientation comprises a combination of directing ionizing output or beams at an internal lesion as a result of movement selected from the group consisting of rotation around an isocenter in a single plane or multiple planes, static or arcing beams shifted closer or further from the skin and lesion, non-coplanar delivery from any direction external to a body originating in a concentric sphere around a targeted lesion, or altering the radiation source's distance from the lesion along radials originating in the targeted lesion;
d. wherein the energy is delivered non-invasively without employing a device or mechanism to cut, puncture or traumatically penetrate skin or other surface, and without an open surgical procedure;
e. wherein an energy-emitting source is aligned in a localized, or circumferential, or ring or spherical distribution around a patient, as a stand-alone arrangement following radials originating in a treated tumor, or in a surface conforming layout, or moved by a robot and not requiring a circular gantry;
f. wherein a phased array of positions of the ionizing energy source is electronically or computer controlled, to shift and contour its output or beams in different directions by mechanically moving the source in space, for discrete and localized delivery of energy dose to a given depth or lateral position in the body with a narrow or focused distribution in a lesion;
g. wherein the resulting contoured energy output or beams need not be isocentric, but are converging electromagnetic kV energy beams and deposit energy dose in a localized volume of interest to treat pathologic tissue;
h. wherein the conforming volume of prescribed x-ray dose is discrete, encompassing and customized to a detrimental phase or lesion within the body, thereby enhancing restoration by controlling parameters to a normal physiologic range and thus enabling a healthy transition state;
i. electronic or computerized controls for source and system operation with a connected power supply and cooling;
j. imaging for planning and guidance, a computer with treatment planning software, which can upload images for planning and guidance, and is connected to an information system with record and verify software;
k. a medium storing computer-executable process steps to reconstruct medical images and calculate and optimize therapeutic effects of the localized treatment with ionizing kV radiation;
l. a treatment table or chair capable of movement in three dimensions;
m. an ionizing-radiation, treatment-localizing agent to treat diseased tissue in response to received radiation energy; and
n. real-time, non-invasive x-ray source beam output measurement.

2. A device comprising:
a. a mechanically distributed energy radiating source, which is external and around a body, or head, to treat and image pathologic lesions;
b. wherein the radiated energy is ionizing, ablating electromagnetic kV ranging from 100-350 kVp;
c. wherein the radiated energy source's orientation comprises a combination of directing ionizing output or beams at an internal lesion as a result of movement selected from the group consisting of rotation around an isocenter in a single plane or multiple planes, static or arcing beams shifted closer or further from the skin and lesion, non-coplanar delivery from any direction external to a body originating in a concentric sphere around a targeted lesion, or altering the radiation source's distance from the lesion along radials originating in the targeted lesion;
d. wherein the energy is delivered non-invasively without employing a device or mechanism to cut, puncture or traumatically penetrate skin or other surface, and without an open surgical procedure;
e. wherein an energy-emitting source is aligned in a localized, or circumferential, or ring or spherical distribution around a patient, as a stand-alone arrangement following radials originating in a treated tumor, or in a surface conforming layout, or moved by a robot and not requiring a circular gantry;
f. wherein a phased array of positions of the ionizing energy source is electronically or computer controlled, to shift and contour its output or beams in different directions by mechanically moving the source in space, for discrete and localized delivery of energy dose to a given depth or lateral position in the body with a narrow or focused distribution in a lesion;
g. wherein the resulting contoured energy output or beams need not be isocentric, but are converging electromagnetic kV energy beams and deposit energy dose in a localized volume of interest to treat pathologic tissue;
h. wherein the conforming volume of prescribed x-ray dose is discrete, encompassing and customized to a detrimental phase or lesion within the body, thereby enhancing restoration by controlling parameters to a normal physiologic range and thus enabling a healthy transition state;
i. electronic or computerized controls for source and system operation with a connected power supply and cooling;
j. a medium storing computer-executable process steps to reconstruct medical images and calculate and optimize therapeutic effects of the localized treatment with ionizing kV radiation; and
k. real-time, non-invasive x-ray source beam output measurement.

3. The device according to claim 2, further comprising:
a. adjustable electronics to optimally contour different locations, shapes, sizes, frequency and timing, and other treatment related parameters of the energy output or beams;
b. wherein a treatment volume can be maintained or adjusted to cover specific pathology, anatomic and functional structures or regions of interest;
c. wherein a resulting adjustable radiation field size covers an internal treatment volume having diameters ranging from millimeters to a plurality of centimeters; and
d. wherein a medium storing computer-executable process steps adjusts the mechanism's radiation emission for treatment and imaging.

4. The device according to claim 2, further comprising:
a. robotic interfaces for orienting and directing electromagnetic waves propagating in space, and resulting secondary electrons flowing through a body, are connected to an integration software of a system;
b, wherein the robot may or may not position an emitting x-ray source around an isocenter; and
c. overlapping directed waves do not touch at a patient's skin but do build up energy deposition in a selected deep-seated volume, which has been delineated beforehand by imaging or functional evaluation, to receive an enhanced dose of kV energy or radiation.

5. A method comprising:
a. effective and deliberate targeting to non-invasively deliver ionizing, ablating, kV electromagnetic energy beams to specific volumes of tissue or locations inside a body or head, wherein the energy targeting is measured and evaluated in real time with imaging, fiducial localization or other functional registration;
b. wherein the ionizing kV energy output is effectively directed according to optimized dose gradient vectors towards a desired location, specific or selected volume, and energy waves traveling towards undesired locations, such as healthy tissue, are quenched;
c. obtaining an original condition, state, conformation, functionality or phase in the body by introducing adequate localized energy to a pathologically altered tissue, network or system;
d. wherein the energy is focused, enhanced and localized energy, and reverses or destroys malignant tissue thereby enabling restoration to their native, normal conformation;
e. wherein the beams' variables and parameters, which are optimized for a three-dimensional gradient field, include but are not limited to, energy, spectrum, filtration, field size, location and orientation in stereotactic space or volume, source-to-skin distance (SSD), generating amperage (mA) and time of delivery, beam shape and collimation, and number and location of beam overlaps, consideration of attenuation for a given beam energy and effects of the inverse square law (1/r\hat{ }2) and inverse cube law (1/r\hat{ }3);
f. wherein the variables and parameters of the focused, enhanced and localized ionizing dose or other energy deposition are optimized as gradient vectors or gradient vector fields;
g. wherein the multiple beams, considered as radial vectors or steradian-associated vectors herein, are focused towards a location targeted in space and time, to deliver effective low-energy ionizing teletherapy;
h. inducing a salutary effect in a diseased tissue comprising a combination of enhancing degradation, elimination, enhancing immune processing and antigen presentation of peptide elements; and
i. wherein the induced effects of focused, enhanced and localized ionizing energy deposition further comprise a combination of enhancing tissue repair and removing tumors internally, each treating a diseased tissue selected from the group consisting of the head, body and other anatomy.

6. The method according to claim 5 wherein:
a. distributing energy radiating source orientations positioned, externally and around a body, including head and central nervous system, to treat and image pathologic lesions;
b. radiating an electromagnetic energy that is ionizing, and ablating;
c. wherein the radiated energy comprises a combination of ionizing output or beams selected from the group consisting of a range of dose gradients used for optimization to maximize function and efficacy;
d. delivering the energy non-invasively without employing a device or mechanism to cut, puncture or traumatically penetrate skin or other surface, and without an open surgical procedure;
e. aligning the energy-emitting source with a target lesion using a robot in a localized, or circumferential, or ring or spherical distribution around a patient, or in a surface conforming route as a stand-alone arrangement;
f. employing an array of the ionizing energy source positions, electronically or computer controlled, to shift and contour their output in different directions by automating and moving the sources, for discrete and localized delivery of energy dose to a given depth or lateral position in the body with a narrow or focused distribution;
g. contouring energy output or beams to concentric, converging electromagnetic energy beams and depositing energy dose in a localized volume of interest to treat pathologic tissue;
h. controlling the sources electronically or with computers, and operating with a connected power supply and cooling; and
i. depositing energy dose locally by shifting and contouring energy output, or beams, for treatment comprising a combination of pathologic lesions, each having a detrimental impact selected from the group consisting of cancer, cancerous and benign tumors and mass effects.

7. The method according to claim 6 further comprising:
a. focusing the output of an array of x-ray source positions on a diseased volume of anatomy to ablating levels in a range of 18 Gy or greater in single fractions, at 24-hour intervals;
b. wherein the positions are distributed along the radials for different spherical volumes emanating from a targeted lesion within normal anatomy;
c. wherein the anatomy comprises a combination of sites selected from the group consisting of head, body or extremities;
d. targeting a lesion within a body by employing volumetrically discrete energy dose deposition to beneficially damage lesions;
e. wherein the conforming volume of prescribed x-ray dose is discrete, encompassing and customized to a detrimental phase or lesion within the body, thereby enhancing restoration by controlling parameters to a normal physiologic range and thus enabling a healthy transition state; and
f. wherein a resulting adjustable radiation field size covers an internal treatment volume having diameters ranging from millimeters to a plurality of centimeters.

8. The method according to claim 7 further comprising:
a. arraying x-ray source positions around a pathologic site to focus and enhance kV range energy within an optimized portion of steradians of dose distribution;
b. wherein optimized steradians of dose distribution are centered in a sphere of possible source positions in a surface area equal to the radius squared;
c. treating with beams along the radii of optimized radiation dose gradients, which diverge from a common center located in a target;
d. wherein the beams' variables and parameters, which are optimized for a three-dimensional gradient field, include but are not limited to, energy, spectrum, filtration, field size, location and orientation in stereotactic space or volume, source-to-skin distance (SSD), generating amperage (mA) and time of delivery, beam shape and collimation, and number and location of beam overlaps, consideration of attenuation for a given beam energy and effects of the inverse square law (1/r\hat{ }2) and inverse cube law (1/r\hat{ }3); and
e. implementing the variables and parameters with automated mechanisms such as robots.

9. The method according to claim 6 wherein:
a. treating diseased tissue with a ionizing-radiation, treatment-localizing agent, wherein the agent becomes active in response to received ionizing-radiation energy;
b. wherein the ionizing-radiation, treatment-localizing agents are selected from the group consisting of liposomes, which carry drugs, biologics, immune-modulating compounds, contrast agents; and
c. targeting a lesion within the body by employing volumetrically discrete energy dose deposition to beneficially manipulate the lesion with a therapeutic compound therein.

10. The method according to claim 5 further comprising:
a. employing computational algorithms to optimize dose gradients by adjusting system controls and parameters;
b. wherein the controls and parameters are modified for case-specific treatment protocols;
c. using independent units, or stand-alone technology, to build a more complex structure and functionality;
d. wherein enabling delivery of more advantageous kV dosimetry derived from vector optimized treatment configurations; and
e. wherein beam path as well as source orientation and distance are included.
